(19) United States
(12) Patent Application Publication
(10) Pub. No.: US 2019/0223964 A1
(43) Pub. Date: Jul. 25, 2019

(54) SYSTEM FOR CAMERA CONTROL IN ROBOTIC AND LAPAROSCOPIC SURGERY

(71) Applicants: QATAR FOUNDATION FOR EDUCATION, SCIENCE AND COMMUNITY DEVELOPMENT, Washington, DC (US); HAMAD MEDICAL CORPORATION, Doha (QA)

(72) Inventors: NIKHIL V. NAVKAR, DOHA (QA); JULIEN ANTOINE ABINAHED, DOHA (QA); SHIDIN BALAKRISHNAN, DOHA (QA); ABDULLA AL-ANSARI, DOHA (QA)

(73) Assignees: QATAR FOUNDATION FOR EDUCATION, SCIENCE AND COMMUNITY DEVELOPMENT, DOHA (QA); HAMAD MEDICAL CORPORATION, DOHA (QA)

(21) Appl. No.: 16/317,324

(22) PCT Filed: Jul. 13, 2017
(86) PCT No.: PCT/US2017/041874

§ 371 Date: Jan. 11, 2019

Related U.S. Application Data
(60) Provisional application No. 62/361,962, filed on Jul. 13, 2016.

(51) Int. Cl. A61B 34/30; A61B 90/00; A61B 34/20; A61B 34/00

(52) U.S. Cl. CPC A61B 34/30; A61B 90/361; A61B 90/37; A61B 2017/00216; A61B 34/74; A61B 2034/2055; A61B 2034/302; A61B 34/20

(57) ABSTRACT

The system for camera control in robotic and laparoscopic surgery (100) includes a head tracking system (130) for tracking movements of an operator's head during laparoscopic surgery, a robotic laparoscope holder (110) operatively engaged to a laparoscope (200), an interface workstation (120) having a processor (540) and inputs connecting the sensor signal and the servo control system (515) signals to the processor (540), and a clutch switch (180) connected to the processor (540) for activating and inactivating the interface workstation (120). The head tracking system (130) includes at least one optical marker (415) to be worn on the operator's head and an optical tracker (410) for detecting movement of the at least one optical marker (415) and transmitting a corresponding sensor signal. The laparoscope includes an articulating distal portion (214), a tip (216), and a camera (150) disposed at the tip (150).

### TECHNICAL FIELD

The present invention relates to a system for laparoscopic surgery, and particularly to a system for controlling a camera attached to a laparoscope by head movements of the surgeon.

### BACKGROUND ART

Typically in a classical laparoscopic surgery setting, a medical professional operates on a tissue using instruments inserted through small incisions. The operating field inside a patient's body is visualized using a camera attached to a laparoscope. During a laparoscopic procedure, both hands of the medical professional are usually occupied with laparoscopic instruments. As such, an experienced assistant is required to maneuver the laparoscope and continuously provide necessary visualization of the operating field to the surgeon. Since the medical professional does not have direct control over the visualization of the operative field, miscommunications between the surgeon and the assistant may occur, leading to complications and increased operating time. Apart from miscommunication, the assistant is subject to fatigue, distractions, and natural hand-tremors that may result in abrupt movement of the operating field on the display.

In an effort to overcome these challenges, robotic laparoscope holders have been used by medical professionals to maintain direct control of the operative field. Typically, the medical professional controls camera movement by providing the robotic laparoscope holder a set of maneuver commands, including tilting, panning, insertion/retraction, rotation and angulation. For such robotic laparoscope holders, the medical professional must first mentally compute the position and orientation of the entire laparoscope to focus the camera at a desired position, and then specify the sequence of maneuvers through an interface, such as a voice-controlled interface, to move the laparoscope. Thus, the interface currently used for the control of these robotic devices requires the surgeon to give a discrete set of commands to focus the camera on a desired location, such as tile-up, tilt-up, pan-right, pan-right, tilt-up, tilt-up, pan-right, tilt-up, angulate, rotate. This can result in poor human-in-the-loop interaction with the robotic laparoscope holder. Further, the incision point acts as a fulcrum for the laparoscope, thereby causing scaling and inversion of movements, as well as making the maneuvering of the camera disposed at the distal end of the laparoscope challenging, especially in the case of articulated and angulated laparoscopes.

Thus, a system for camera control in robotic and laparoscopic surgery solving the aforementioned problems is desired.

### DISCLOSURE OF INVENTION

The system for camera control in robotic and laparoscopic surgery includes a head tracking system for tracking movements of an operator's head during laparoscopic surgery, a robotic laparoscope holder operatively engaged to a laparoscope, an interface workstation having a processor and inputs connecting the sensor signal and the servo control system signals to the processor, and a clutch switch connected to the processor for activating and inactivating the interface workstation. The head tracking system includes at least one optical marker to be worn on the operator's head and an optical tracker for detecting movement of the at least one optical marker and transmitting a corresponding sensor signal. The laparoscope includes an articulating distal portion, a tip, and a camera disposed at the tip.

These and other features of the present invention will become readily apparent upon further review of the following specification and drawings.

### BRIEF DESCRIPTION OF DRAWINGS

FIG. 1 is a diagram of a generalized system for camera control in robotic and laparoscopic surgery, according to the present disclosure.

FIG. 2A is an environmental, side view a laparoscope extending through a cannula and into a patient's body, according to the present disclosure.

FIG. 2B is an environmental, side view of a robotic laparoscopic holder holding the laparoscope during a laparoscopic surgical procedure, according to the present disclosure.

FIG. 3A illustrates an articulated laparoscope that may be utilized in connection with the system for camera control in robotic and laparoscopic surgery, according to the present disclosure.

FIG. 3B illustrates an angulated laparoscope that may be utilized in connection with the system for camera control in robotic and laparoscopic surgery, according to the present disclosure.

FIG. 3C illustrates a zero-degree laparoscope that may be utilized in connection with the system for camera control in robotic and laparoscopic surgery, according to the present disclosure.

FIG. 4A illustrates a head tracking system for use in connection with the system for camera control in robotic and laparoscopic surgery, according to the present disclosure.

FIG. 4B illustrates a one-to-one mapping between a head frame and a camera frame, according to the present disclosure.

FIG. 4C illustrates a plurality of optical markers arranged in a configuration, according to the present disclosure.

FIG. 4D illustrates the plurality of optical markers arranged in an alternative configuration, according to the present disclosure.

FIG. 4E illustrates the plurality of optical markers arranged in another configuration, according to the present disclosure.

FIG. 4F illustrates the plurality of optical markers arranged in another configuration, according to the present disclosure

FIG. 5 is a diagram of a generalized system of an interface workstation for use in connection with the system for camera control in robotic and laparoscopic surgery, according to the present disclosure.

FIG. 6A illustrates an articulation angle and an articulated section length of an articulating distal portion of an articulated laparoscope, according to the present disclosure.

FIG. 6B illustrates the insertion of the articulating distal portion of the articulated laparoscope within the cannula along the ‘Z’ axis, according to the present disclosure.

FIG. 6C illustrates the articulation of the articulating distal portion of the articulated laparoscope about the ‘Z’ axis, according to the present disclosure.

FIG. 6D illustrates the articulation of the articulating distal portion of the articulated laparoscope along the ‘Z’ axis, according to the present disclosure.

FIG. 6E illustrates an angulated laparoscope, according to the present disclosure.

FIG. 6F illustrates the insertion of the shaft of the angulated laparoscope within the cannula along the ‘Z’ axis, according to the present disclosure.

FIG. 6G illustrates the articulation angle of the angulated laparoscope about the ‘Z’ axis, according to the present disclosure.

FIG. 6H illustrates the articulation of the camera of the angulated laparoscope along the ‘Z’ axis, according to the present disclosure.

FIG. 7A illustrates a view direction of a camera disposed at a tip end of the articulating distal portion of the articulated laparoscope, according to the present disclosure.

FIG. 7B illustrates the computation of the camera frame for the articulated laparoscope, according to the present disclosure.

FIG. 7C illustrates the computation of the articulating point of the articulated laparoscope to reposition the camera frame, according to the present disclosure.

FIG. 7D illustrates the computation of the angle by which the camera frame is rotated along the ‘Z’ axis of the articulated laparoscope, according to the present disclosure.

FIG. 7E illustrates a view direction of a camera disposed at a tip end of the angulated laparoscope, according to the present disclosure.

FIG. 7F illustrates the computation of the laparoscope angulation angle for the angulated laparoscope, according to the present disclosure.

FIG. 7G illustrates the computation of the incision frame for the angulated laparoscope, according to the present disclosure.

FIG. 7H illustrates the computation of the camera frame for the angulated laparoscope, according to the present disclosure.

FIG. 8A is a flowchart illustrating the steps of a method for utilizing the system for camera control in robotic and laparoscopic surgery, according to the present disclosure.

FIG. 8B is a flowchart illustrating the steps of a method for utilizing the system for camera control in robotic and laparoscopic surgery, according to the present disclosure.

FIG. 8C is a flowchart illustrating the steps of a method for utilizing the system for camera control in robotic and laparoscopic surgery, according to the present disclosure.

FIG. 8D is a flowchart illustrating the steps of a method for utilizing the system for camera control in robotic and laparoscopic surgery, according to the present disclosure.

FIG. 9A illustrates the system for camera control in robotic and laparoscopic surgery having at least one robotic arm, according to the present disclosure.

FIG. 9B illustrates the system for camera control in robotic and laparoscopic surgery having one robotic arm, according to the present disclosure.

FIG. 9C illustrates the system for camera control in robotic and laparoscopic surgery having one flexible robotic arm, according to the present disclosure.

Similar reference characters denote corresponding features consistently throughout the attached drawings.

### BEST MODES FOR CARRYING OUT THE INVENTION

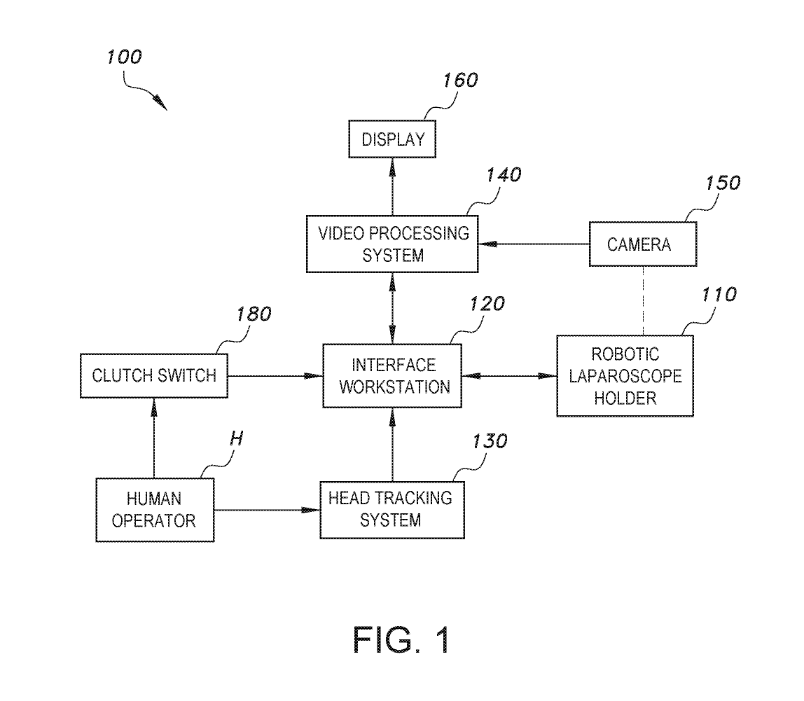

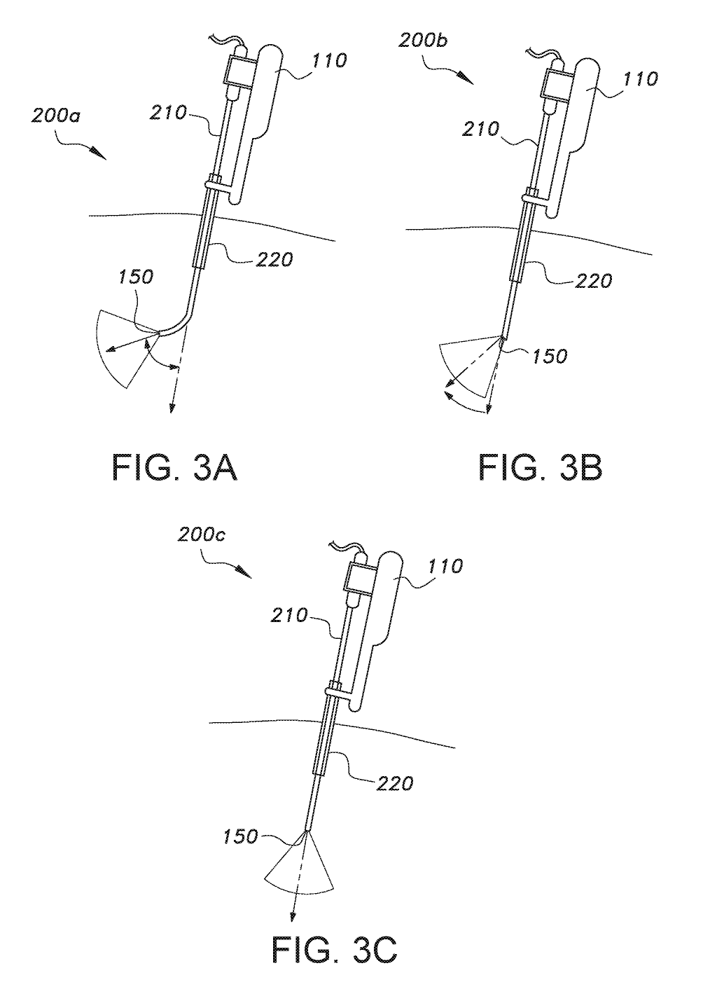

Referring to FIGS. 1 through 9C, a system for camera control in robotic and laparoscopic surgery 100 is generally illustrated. The system 100 is configured to track movement of the head of a human operator H (e.g., surgeon) during a laparoscopic surgical procedure and move the camera 150 in a direction that corresponds to the movement of the head. The system 100 can be a software module running on an interface workstation 120. The system 100 includes a robotic laparoscope holder 110, a laparoscope, such as an articulated laparoscope 200a (FIG. 3A), an angulated laparoscope 200b (FIG. 3B) or a zero degree laparoscope 200c (FIG. 3C), selectively connected to the laparoscope holder, such as via a laparoscope adapter 205, a head tracking system 130 in communication with the interface workstation, a video processing system in communication with the interface workstation 120, a display 160 in communication with the video processing system 140, and a clutch in communication with the interface workstation. The laparoscope 200a-200c includes a shaft 210, such as an elongated shaft, having a proximal portion 212 and a distal portion 214, such as an articulating distal portion in the articulated laparoscope 200a (FIG. 3A). The distal portion 214 includes a tip end 216 and a camera 150 disposed at the tip end 216.

The interface workstation 120 receives and/or sends commands to the head tracking system 130, the video processing system 140, the robotic scope holder 110, and the clutch switch 180. The human operator H selects the scope type and model from a list available on the interface workstation before the surgery.

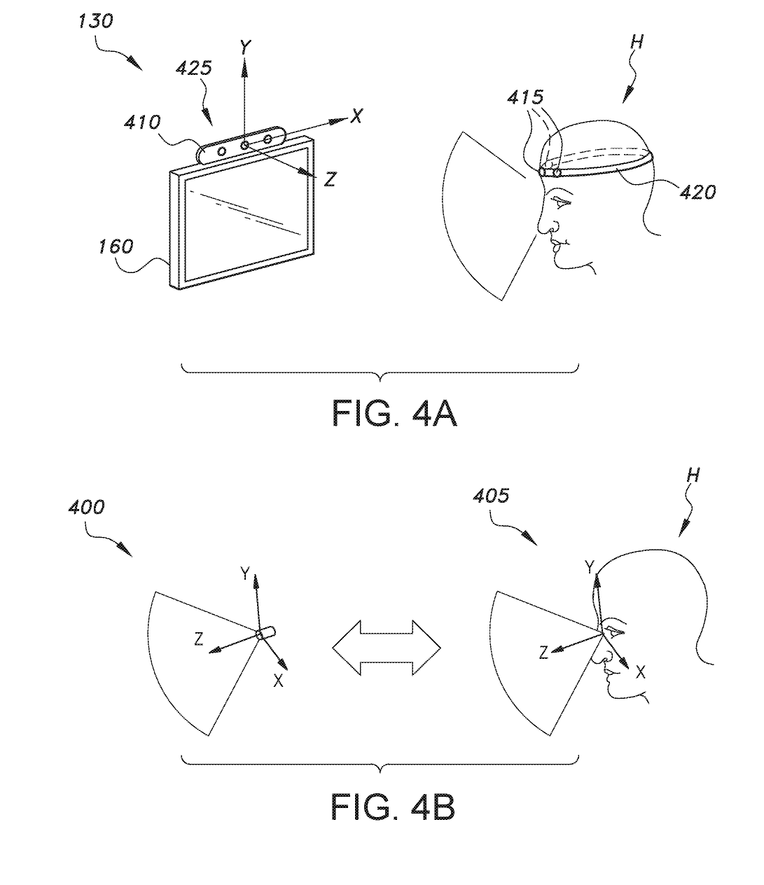

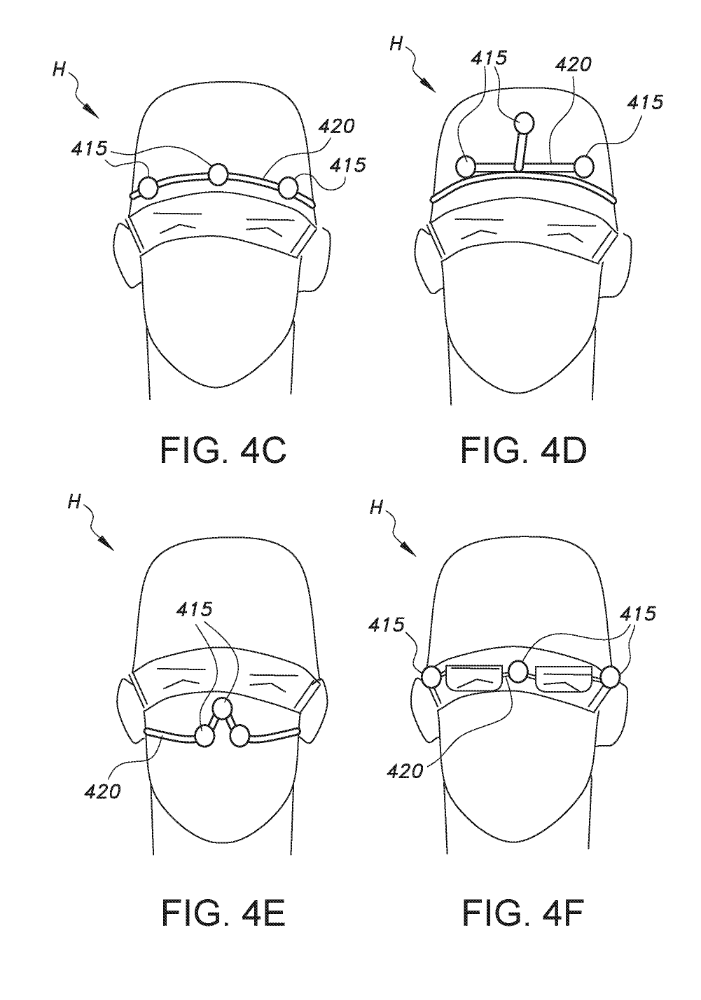

The head tracking system 130 includes one or more optical markers 415, e.g., three optical markers 415, that are attachable to the head of the human operator H, and an optical tracker 410 that is configured to track the spatial location of the optical markers 415 and to translate or transform this information into a virtual head-frame representing the position and orientation of the operator's head (FIGS. 4A-4F). The optical markers 415 can be arranged in a manner that allows triangulating the position/orientation of the head in a three-dimensional space. The head tracking system 130 is configured for communicating the spatial orientation of the operator's head to the interface workstation 120 to facilitate control of the movement/positioning of the laparoscope 200 during the surgical procedure. The optical tracker 410 can be positioned at any suitable location, such as on the display 160, as illustrated in FIG. 4A. The one or more optical markers 415 can be attached at a predetermined spot on a band 420 worn on the medical professional's head H. The head tracking system 130 can smooth and scale the head motion based on parameters set preoperatively by the operator during calibration. The head tracking system 130 may be any conventional, off-the-shelf head tracking system, such as the TrackIR5 from NaturalPoint.

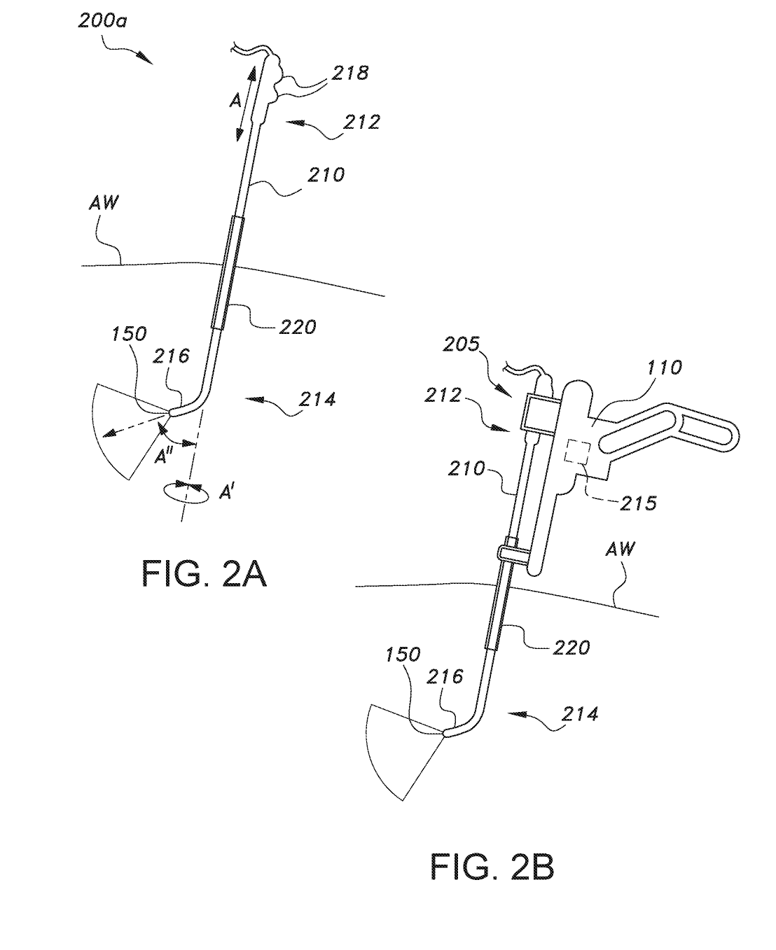

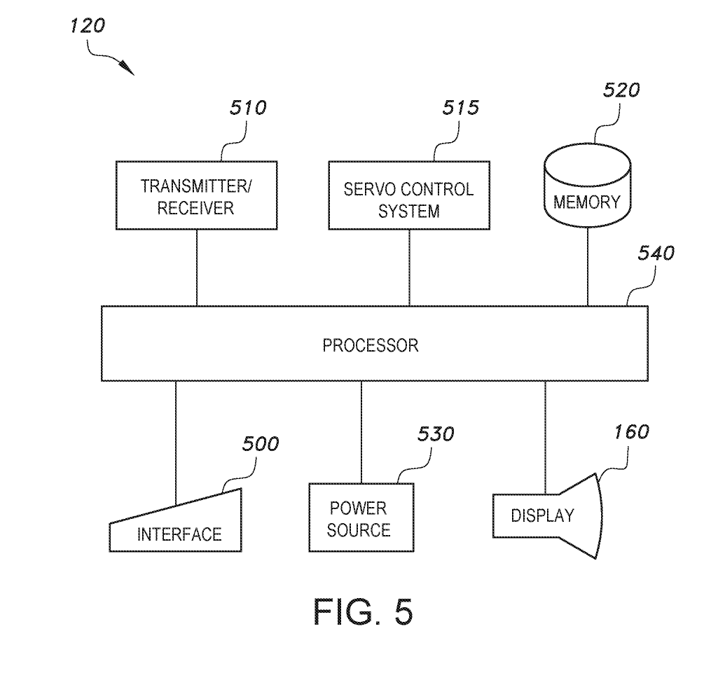

The robotic scope holder 110 can be a robot configured to move the laparoscope 200a-200c. The laparoscope 200a-200c can be placed on the robotic scope holder 110 using a scope adaptor 205 (FIGS. 2A-2B). The robotic scope holder 110 may also be configured to hold a trocar/cannula through which the scope is inserted. The interface workstation sends a command to actuate the robotic scope holder 110. The actuation command is in the form of a configuration parameter representing the robot's end effector position and orientation. The actuation allows the scope's camera to be placed in a particular orientation and position as specified by the operator. The robotic laparoscope holder 110 includes at least one servomechanism 215, commonly known in the art, for moving and manipulating the robotic laparoscope holder 110 and the camera 150 as directed by the interface workstation 120. The at least one servomechanism 215 is configured for receiving actuating signals and for sending signals reporting the status of the camera 150 to a servo control system 515 (FIG. 5), described in detail below.

The articulating distal portion 214 of the shaft 210 is configured for insertion into the patient's body, such as through a cannula 220 inserted through the patient's abdominal wall AW. A camera frame or virtual frame 400 is defined at the tip of the scope to identify the position and orientation of the camera. As the camera frame 400 moves in a three-dimensional (3D) space, the laparoscope 200a-200c follows the motion of the camera frame 400. There is a one-to-one direct mapping of the surgeon's head movement (defined by head-frame) to the motion of the camera. The camera output is rendered on the display 160. For example, a video stream of the operating field from the scope camera 150 is provided to the video processing system 140 which rotates the video stream of the operating field with superimposed information which is then provided to the display 160 for operator viewing.

The proximal portion 212 of the laparoscope 200 includes a plurality of knobs 218. As illustrated by arrow A, each knob 218 may selectively extend and retract the articulating distal portion 214 in into or out of the incision. The knobs 218 can be configured to maneuver the articulating distal portion 214 of the laparoscope 200 within the surgical environment. For example, the knobs 218 may rotate the articulating distal portion 214 of the shaft 210 about the vertical axis, as illustrated by arrow A′, and/or articulate the articulating distal portion 214 of the shaft 210, as illustrated by arrow A″, to reposition the camera 150 within the surgical environment. In case of angulated laparoscope 200b, the rotation along the vertical axis A′ can also be performed directly by rotating the shaft 210 using the robotic laparoscope holder 110.

The clutch switch 180 may be used to activate or deactivate the interface workstation 120 and, in turn, the system 100. The clutch switch 180 may act as an ‘ON’ and ‘OFF’ switch and, as such, may be any suitable type of activation switch, such as a foot pedal or a button, attached to the robotic laparoscope holder 110, or a voice activated receiver. The switching between ‘ON’ and ‘OFF’ allows for ergonomic repositioning of the operator's head H in front of the optical tracker 410.

The band 420 may be any suitable type of band formed from a lightweight, flexible material allowing the band 420 to have a variety of configurations, as illustrated in FIGS. 4C through 4F, which may allow for better communication between the optical markers 415 and the optical tracker 410.

The camera 150 may be any suitable medical grade type of camera adapted for acquiring video images of the surgical environment within the patient's body and for communicating a video stream to the video processing system 140 to show on the display 160. The display 160 may be any suitable type of display, such as a light emitting diode (LED) or liquid crystal display (LCD).

The video processing system rotates the video as requested by the interface workstation. The rotational angle by which the video is rotated at the center of the visualization screen is represented by R_{Screen}(t). It is measured with respect to the “X” axis of an imaginary 2D coordinate system located at the center of the screen and with the axes parallel to the sides of the visualization screen. The angle is measured in degrees.

Any motion of the optical markers placed on the operator's head is tracked by the head tracking system. The head frame 405 or orientation and position of the operator's head is measured with respect to a head tracking base frame 425. The head frame is represented by a 4×4 homogenous transformation matrix, M_{Head-Frame}(t₀), wherein ‘t₀’ represents the time at which the frame was captured. For example, each of the optical markers 415 are arranged in a specific configuration that allows the optical tracker 410 to triangulate the orientation and position of the medical professional's head within a three-dimensional (“3D”) space in real-time. As illustrated in FIG. 4B, the ‘Z’ axis of the head frame 405 may coincide with the medical professional's viewing direction (e.g. the direction of his/her eyes) and the ‘Y’ axis point coincides with the vertical movement, such as in an upward and downward direction, of the medical professional's head H. Accordingly, as the medical professional moves his/her head H (i.e. either roll, yaw, pitch, or along the X, Y, and Z axis) the camera 150 may follow the same motion; thereby, making controlling of the robotic laparoscope holder 110 and, in turn, the laparoscope 200a-200c more intuitive.

If the desired position and orientation of the camera 150 (as per the medical professional's head movements) is not achievable due to certain constraints, the interface workstation 120 may utilize a transformation filter (not shown) to compute feasible positions and orientations for the camera 150 to move. Such constraints, which may be incorporated in various filters, may include: (1) tissue boundaries and (2) unreachable workspaces for the camera 150. The tissue boundaries may be computed from preoperative medical imagining data (e.g. MR scans or CT scans) to avoid impingement of the camera 150 with vital structures. In the case of zero-degree or angulates laparoscopes, the limited degrees of freedom may restrict the motion of the camera 150.

The processing of the video stream produced by the camera 150 may involve rotating the images of the video stream by a predetermined angle. The rotational angle, measured in degrees (°), by which the video is rotated is represented by R_{Screen}(t) and is measured with respect to the “X” axis of an imaginary two dimensional (e.g. 2D) coordinate system located at the center of the display 160, the “X” axis being parallel to the sides of the display 160. As illustrated in FIG. 4B, there is a one-to-one direct mapping of the movement of the medical professional's head within the head frame 405 and the movement of the camera 150 within the camera frame 400. The head tracking system 130 may also smooth and scale the head motion based on the parameters set preoperatively by the medical professional during the calibration process, such that the camera 150 may seamlessly follow the movements defined by the medical professional's head position, easily switch directions, and move smoothly within operating field.

The interface workstation 120 may be a centralized system that sends and receives commands to and from the robotic laparoscopic holder 110, the head tracking system 130, the video processing system 140, and the clutch switch 180. As such, it is to be noted that the interface workstation 120 may represent a standalone computer, computer terminal, portable computing device, networked computer or computer terminal, or networked portable device, and can also include a microcontroller, an application specific integrated circuit (ASIC), or a programmable logic controller (PLC).

Data can be entered into the interface workstation 120 by the medical professional, or sent or received from or by any suitable type of interface 500, such as the robotic laparoscopic holder 110, the head tracking system 130, the video processing system 140, or the clutch switch 180, as can be associated with a transmitter/receiver 510, such as for wireless transmission/reception or for wireless communication for receiving signals from a processor 540 to articulate the articulating distal portion 214 of the laparoscope 200 and to reposition the camera 150.

The interface workstation 120 may include a memory 520 such as to store data and information, as well as program(s), instructions, or parameters for implementing operation of the system 100. The memory 520 can be any suitable type of computer readable and programmable memory, such as non-transitory computer readable media, random access memory (RAM) or read only memory (ROM), for example. The interface workstation 120 can be powered by a suitable power source 530.

The interface workstation 120 provides new configuration parameters to actuate the robotic scope holder and move the laparoscope. The interface workstation 120 also receives current configuration parameters measured from the actuator states of the robotic scope holder. The processor 540 of the interface workstation 120 is configured for performing or executing calculations, determinations, data transmission or data reception, sending or receiving of control signals or commands, such as in relation to the movement of the robotic laparoscope holder 110 and/or the camera 150, as further discussed below. The processor 540 can be any suitable type of computer processor, such as a microprocessor or an ASIC, and the calculations, determinations, data transmission or data reception, sending or receiving of control signals or commands processed or controlled by the processor 540 can be displayed on the display 160. The processor 540 can be associated with, or incorporated into, any suitable type of computing device, for example, a personal computer or a PLC. The display 160, the interface 500, the transmitter/receiver 510, the servo control system 515, the memory 520, the power source 530, the processor 540, and any associated computer readable media are in communication with one another by any suitable type of data bus, as is well known in the art.

The point of reference for the robotic laparoscope holder 110 is generally referred to as a robot base frame 600, which is a fixed reference frame for the entire robotic scope holder 110. As such, any motion/movement of the robotic laparoscope holder 110 may be measured with respect to the robot base frame 600. The robotic laparoscope holder 110 may include a mechanism for holding a trocar for creating an incision in the abdominal wall AW of the patient. The position of the trocar depends upon the surgery and patient position. Once the incision is made before the surgery, the robotic laparoscope holder 110 is manually adjusted by the operator to hold the trocar.

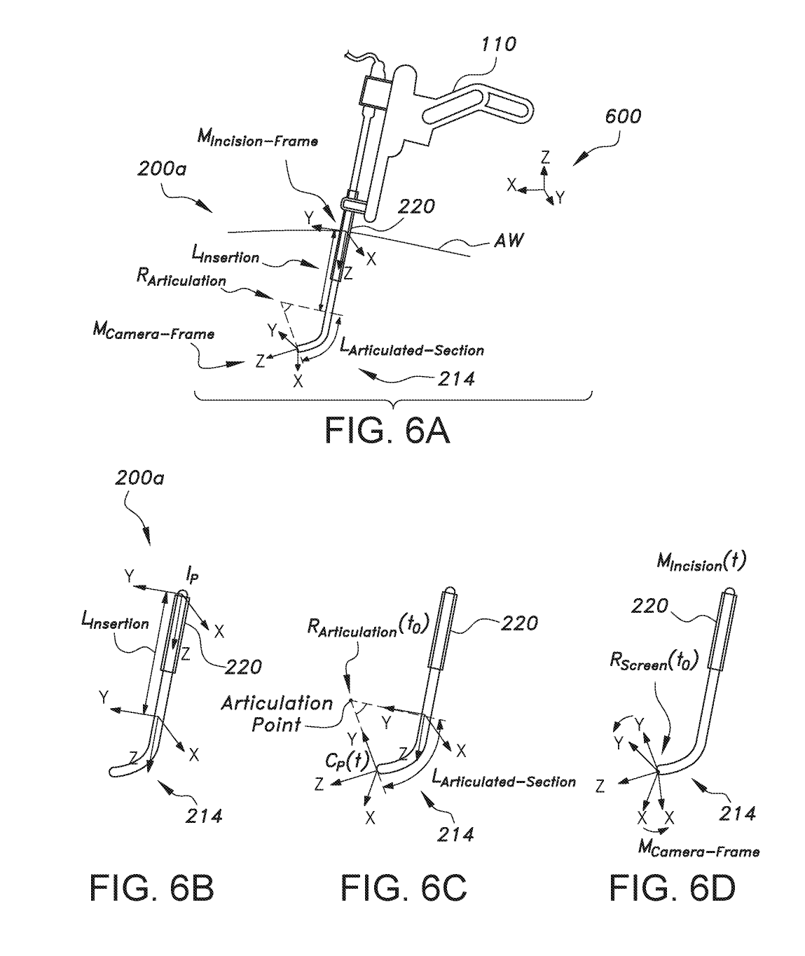

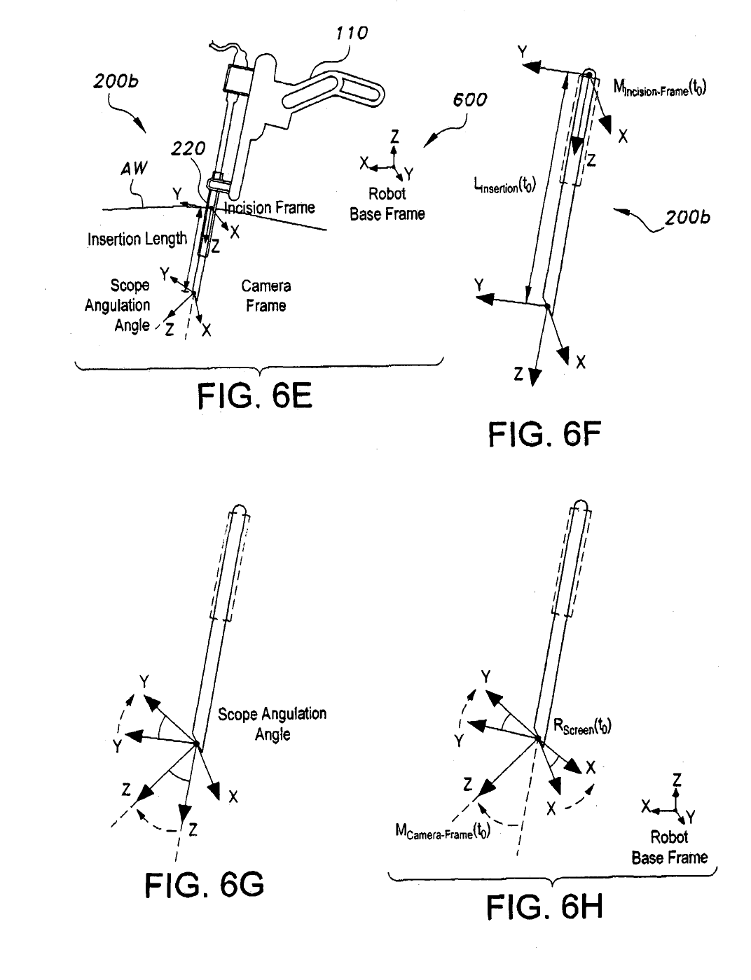

The incision frame for both articulated laparoscopes 200a (FIG. 3A) and angulated laparoscopes 200b (FIG. 3B) is represented by a 4×4 homogenous transformation matrix and is measured with respect to the robot base frame 600, and, as such, may represent the position of the trocar. The origin of the incision frame M_{Incision-Frame} remains stationary as it is the incision point I_{P}. The cannula 220 may be inserted into the body at the incision point I_{P}. As illustrated in FIG. 6A, the ‘Z’ axis represents the direction of the insertion path of the articulating distal portion 214 of the articulated laparoscope 200a, and the direction of the ‘X’ axis is orthogonal to the plane of articulation of the articulating distal portion 214. In case of a zero degree laparoscope 200c (FIG. 3C), the “X” axis is parallel to the axis defined by the robotic laparoscope holder 110.

For the articulated laparoscope 200a (FIG. 3A), the articulated section length L_{Articulated-Section} represents the length of the articulating distal portion 214 of the shaft 210. While the articulated section length L_{Articulated-Section}, remains constant, for a specific articulated laparoscope 200a, the articulated section length L_{Articulated-Section} may vary from one type of laparoscope to another. For computation of parameters for the laparoscope, the articulation of the articulating distal end 214 (e.g. the inward and outward bending movement) occurs in one plane. The articulation angle R_{Articulation} of the articulating distal portion 214 of the shaft 210 of the articulated laparoscopes 200a (FIG. 3A) is a function of the movement of each knob 218. Accordingly, there is a mapping between the movement of each knob 218 and the articulation angle R_{Articulation} of the articulating distal portion 214 of the shaft 210, the articulation angle R_{Articulation} being measured in degrees (°). It is to be noted that that the angulated laparoscope 200b (FIG. 3B) does not have an articulation angle R_{Articulation}.

The insertion length L_{Insertion} for both the articulated laparoscope 200a (FIG. 3A) and the angulated laparoscope 200b (FIG. 3B) is distance between the incision frame M_{Incision-Frame} and the beginning of the distal portion 214. The laparoscope angulation angle for the angulated laparoscope 200b (FIG. 3B) is consistent. The zero degree laparoscope 200c (FIG. 3C), however, has a laparoscope angulation angle equal to zero degrees.

Once positioned in the surgical environment within the patient's body, the camera 150 moves within the camera frame 400. The camera frame 400 describes the position and orientation of the camera 150 at a specific point in time, such as time ‘t’. Accordingly, the camera frame 400 is represented by a 4×4 homogenous transformation matrix, M_{Camera-Frame}. The camera frame 400 is measured with respect to the robot base frame 600 and, as such, represents the position of the camera 150. As illustrated in FIG. 6A, the ‘Z’ axis denotes the viewing direction of the camera 150. If the ‘Z’ direction is aligned with the camera frame 400 and the incision frame M_{Incision-Frame}, the ‘X’ axis of the camera frame 400 will substitute the angle of R_{Screen} to the ‘X’ axis of the incision frame M_{Incision-Frame}, R_{Screen} representing the rotational angle by which the video is rotated at the center of the display 160.

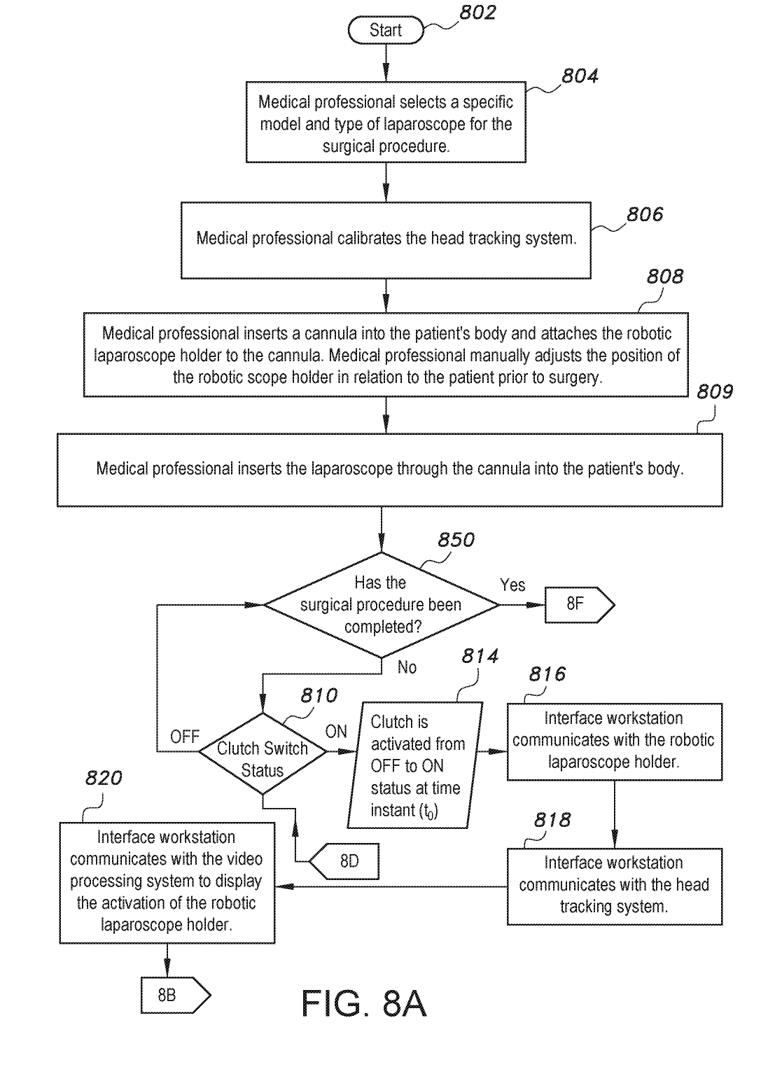

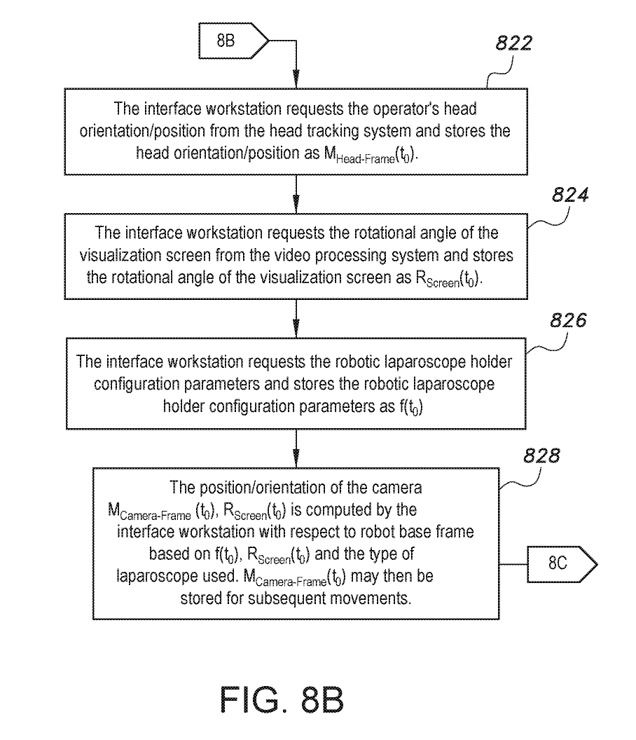

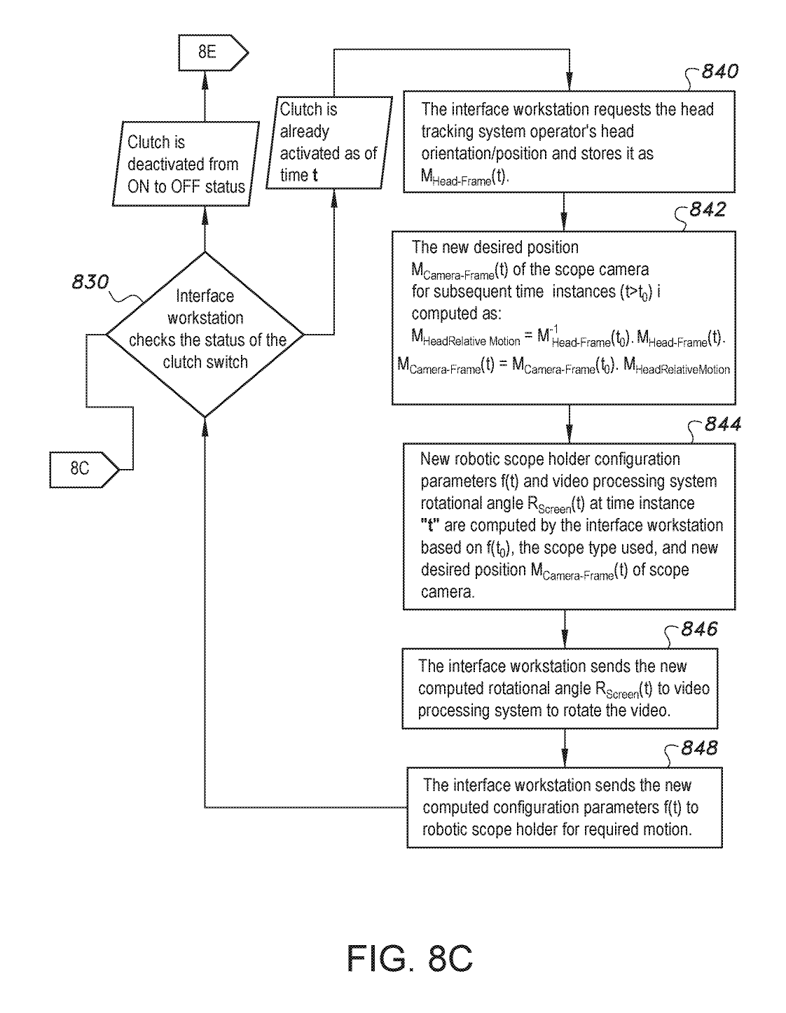

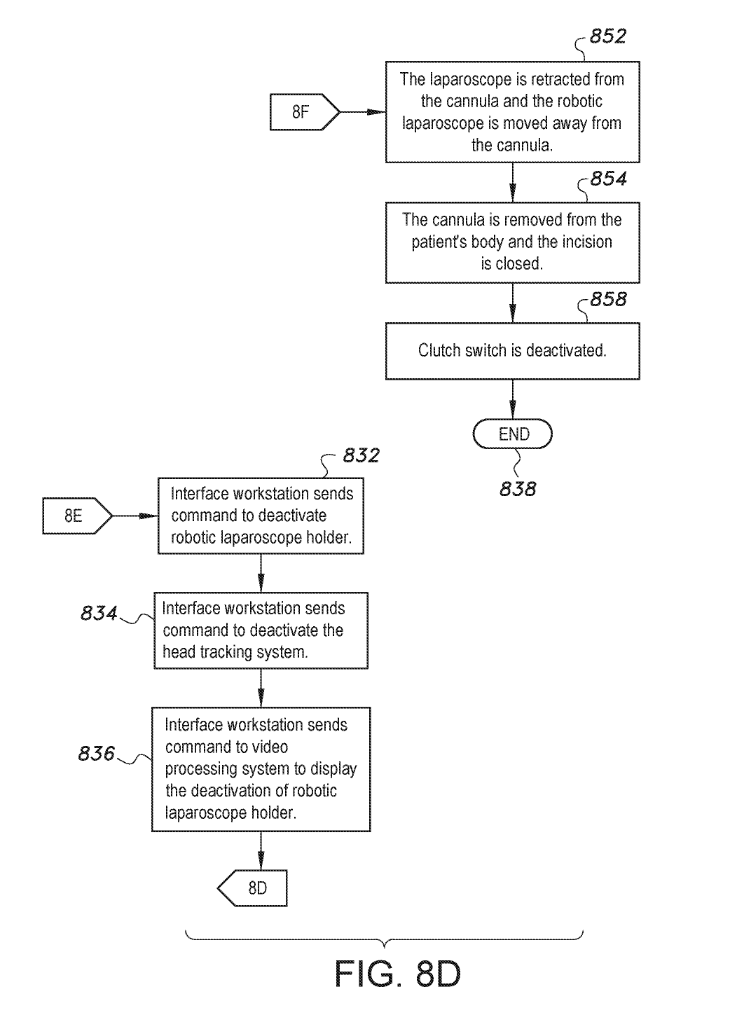

FIGS. 8A-8E illustrates a process flow 800 for the method of utilizing the system for camera control in robotic and laparoscopic surgery 100. To start (Step 802) utilizing the system 100, the medical professional must select a specific type and model of laparoscope 200 (Step 804). The medical professional then needs to calibrate, such as manually calibrate, the head tracking system 130, such that each of the optical markers 415 positioned on the band 420 on medical professional's head H may communicate with the optical tracker 410 of the head tracking system 130 to determine the spatial orientation of the medical professional's head H (Step 806). The medical professional then inserts the cannula 220 through the arterial wall AW of the patient, attaches the robotic laparoscope holder 110 to the cannula 220, and adjusts, such as manually adjusts, the position of the robotic laparoscope holder 110 in relation to the patient's body (e.g. the desired location for the incision) (Step 808). After the robotic laparoscopic holder 100 has been properly positioned, the medical professional attaches the laparoscope 200a-200c to the laparoscope adaptor 205. Subsequently, the medical practitioner inserts the shaft 210 of the laparoscope 200a-200c through the cannula 220 into the patient's body (Step 809).

Once the clutch switch 180 is turned “ON,” the interface workstation 120 may communicate with the head tracking system 130 (Step 816) to activate the head tracking system 130 and determine the spatial location (e.g. the position and orientation) of the medical professional's head H within the head frame 405, with the robotic laparoscopic holder 110 (Step 818) to activate the robotic scope holder 110 and, in turn, to activate the laparoscope 200, and with the video processing system 140 (Step 820) to activate the video processing system 140 and display the activation of the robotic laparoscopic holder 110 on the display 160.

Once the video processing system 140 is activated, the camera 150 may stream real-time video of the operating field through the video processing system 140, such that the new rotational angle(s) may be displayed on the display 160, such as superimposed on the operating field seen on the display 160, which may allow the medical professional to view the operating field, along with the spatial orientation of his/her instruments on the display 160.

The time at which the clutch switch 180 is activated is generally referred to as (t₀). Once activated, the interface workstation 120 requests the medical professional's head orientation/position from the head tracking system 130 and stores the medical professional's head orientation/position as M_{Incision-Frame}(t₀) (Step 822). The interface workstation 120 also requests the rotational angle of the visualization screen and stores the rotational angle as R_{Screen}(t₀) (Step 824). Further, the interface workstation 120 requests the robotic laparoscope holder's 110 configuration parameters, (e.g. (<M_{Incision-Frame}(t₀), L_{Insertion}(t₀), R_{Articulation}(t₀)> for articulated laparoscopes 200a and (<M_{Incision-Frame}(t₀), L_{Insertion}(t₀)> for angulated laparoscopes 200b, as well as for the zero-degree laparoscopes 200c, and stores the configuration parameters as f(t₀) (Step 826). These configuration parameters can be communicated between the interface workstation 120 and the robotic laparoscope holder 110, the head tracking system 130, and the camera 150. Further, the configuration parameters are sufficient to define the configuration for the robotic laparoscope holder's 110 degrees of freedom at time (t) for either articulated laparoscopes 200a or angulated laparoscopes 200b. The position/orientation of the camera at time instant “t” is the “camera frame” and is represented by a 4×4 homogenous transformation matrix M_{camera-Frame}(t₀). The camera frame may be computed by the interface workstation 120 with respect to the robot base frame 600 based on f(t₀)=<M_{Incision-Frame}(t₀), L_{Insertion}(t₀), R_{Articulation}(t₀)>, R_{Screen}(t₀) and the type of laparoscope 200a used (articulated in this case) (Step 828) or f(t₀)=<M_{Incision-Frame}(t₀), L_{Insertion}(t₀)>, R_{Screen}(t₀), and the type of laparoscope 200b or 200c used (angulated or zero-degree in this case). The Z axis denotes the viewing direction of the camera. If the Z direction and origin of both the camera frame and incision frame are aligned, the X axis of the camera frame will substitute an angle of R_{Screen} (t) to the X axis of the incision frame. The camera frame M_{Camera-Frame}(t₀) may then be stored as a basis for computing subsequent movements.

The position/orientation of the camera frame M_{Camera-Frame}(t₀) as it relates to the articulated laparoscope 200a may be computed in three steps by applying affine transformations: (1) the incision frame M_{Incision-Frame}(t₀) is translated along the ‘Z’ axis of the incision frame M_{Incision-Frame}(t₀) for a distance equal to the insertion length L_{Insertion}(t₀) (FIG. 6B); (2) the translated incision frame M_{Incision-Frame}(t₀) is rotated by an articulation angle R_{Articulation}(t₀) around a line passing through articulation point and orthogonal to the ‘YZ’ plane (FIG. 6C) (the articulation point Aₚ lies on the same plane as the ‘Y’ and ‘Z’ axis of the translated frame in the center of the arc representing the articulated section of the articulated laparoscope 200a); and (3) the rotated incision frame M_{Incision-Frame}(t₀) is again rotated along the ‘Z’ axis by an angle of R_{Screen}(t₀) to achieve the camera frame M_{Camera-Frame}(t₀) (FIG. 6D).

The position/orientation of the camera frame M_{Camera-Frame}(t₀) as it relates to the angulated laparoscope 200b may be computed in three steps by applying affine transformations: (1) the incision frame M_{Incision-Frame}(t₀) is translated along the ‘Z’ axis of the incision frame M_{Incision-Frame}(t₀) for a distance equal to the insertion length L_{Insertion}(t₀) (FIG. 6F); (2) the translated incision frame M_{Incision-Frame}(t₀) is then rotated by the laparoscope angulation angle along the “X” axis (FIG. 6G); and (3) the rotated incision frame M_{Incision-Frame}(t₀) is again rotated along the ‘Z’ axis by an angle of R_{Screen}(t₀) to achieve the camera frame M_{Camera-Frame}(t₀) (FIG. 6H).

After all the information from the robotic laparoscope holder 110, the head tracking system 130, and the video processing system 140 has been obtained and stored by the interface workstation 120, the interface workstation 120 re-checks the clutch switch 180 to determine whether the clutch switch 180 remains active or whether the clutch switch 180 has been deactivated (Step 830). If the clutch switch 180 has been deactivated, the interface workstation 120 sends a command to the robotic laparoscopic holder 110 to deactivate the robotic laparoscope holder 110 (Step 832). The interface workstation 120 also sends a command to the head tracking system 130 to deactivate the head tracking system 130 (Step 834). Lastly, the interface workstation 120 sends a command to the video processing system 140 to display the deactivation of the robotic laparoscope holder 110 (Step 836). Subsequently, the process flow returns to Step 810 and proceeds as described herein.

If, on the other hand, the clutch switch 180 remains active (e.g. the clutch switch 180 is still turned “ON”), such as from the initial time (t₀), the interface workstation 120 requests the head tracking system operator's head orientation/position and stores it as M_{Head-Frame}(t) (Step 840). The new desired position M_{Camera-Frame}(t) of the camera 160 at subsequent time instances ‘t’ may be calculated by using the following equations:

M_{HeadRelativeMotion}=M⁻¹_{Head-Frame}(t₀)×M_{Head-Frame}(t) (1)

M_{Camera-Frame}(t)=M⁻¹_{Head-Frame}(t₀)×M_{HeadRelativeMotion} (2)

### (Step 842).

After the new desired position M_{Camera-Frame}(t) of the laparoscope camera 200a-200c has been computed, the interface workstation 120 begins to calculate the new robotic laparoscope holder configuration parameters at a subsequent time instances (t), herein (t>t₀) (e.g. f(t)) and the video processing system rotational angle R_{Screen}(t) based on f(t₀), the type of laparoscope used, and the new desired position M_{Camera-Frame}(t) of the camera 150 (Step 844). If the articulated laparoscope 200a is being used the following configuration parameters are used: f(t₀)=<M_{Incision-Frame}(t₀), L_{Insertion}(t₀), R_{Articulation}(t₀)>. If, however, the angulated laparoscope 200b is being used the following configuration parameters are used: f(t₀)=<M_{Incision-Frame}(t₀), L_{Insertion}(t₀)>.

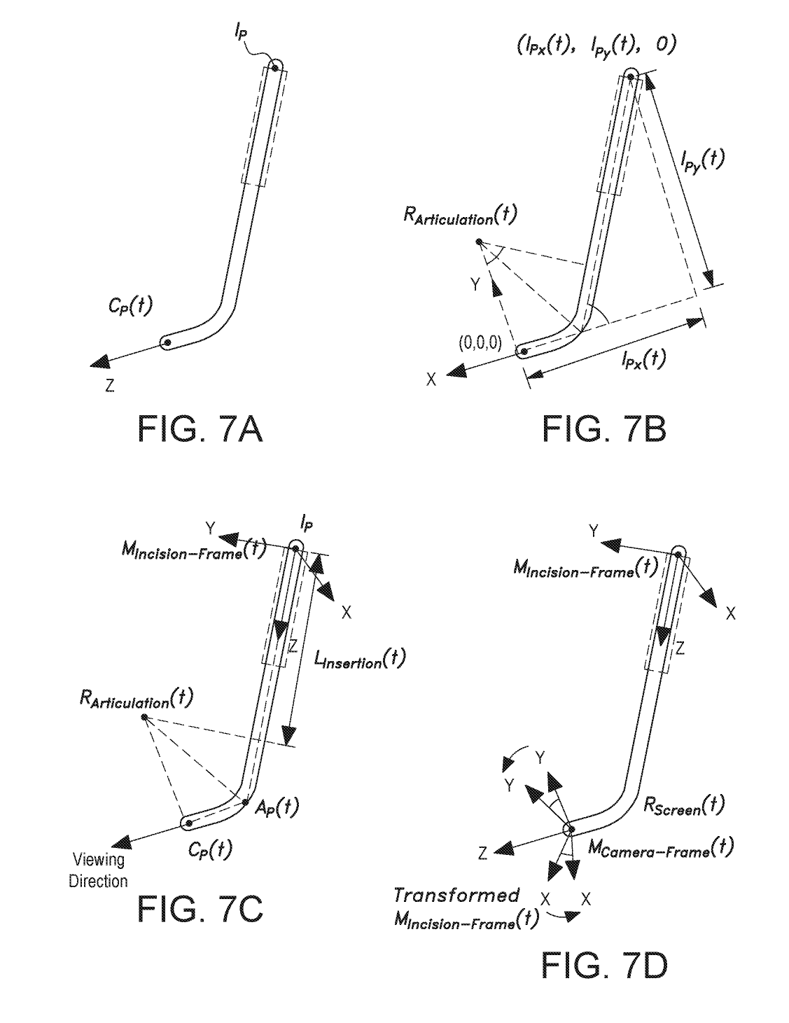

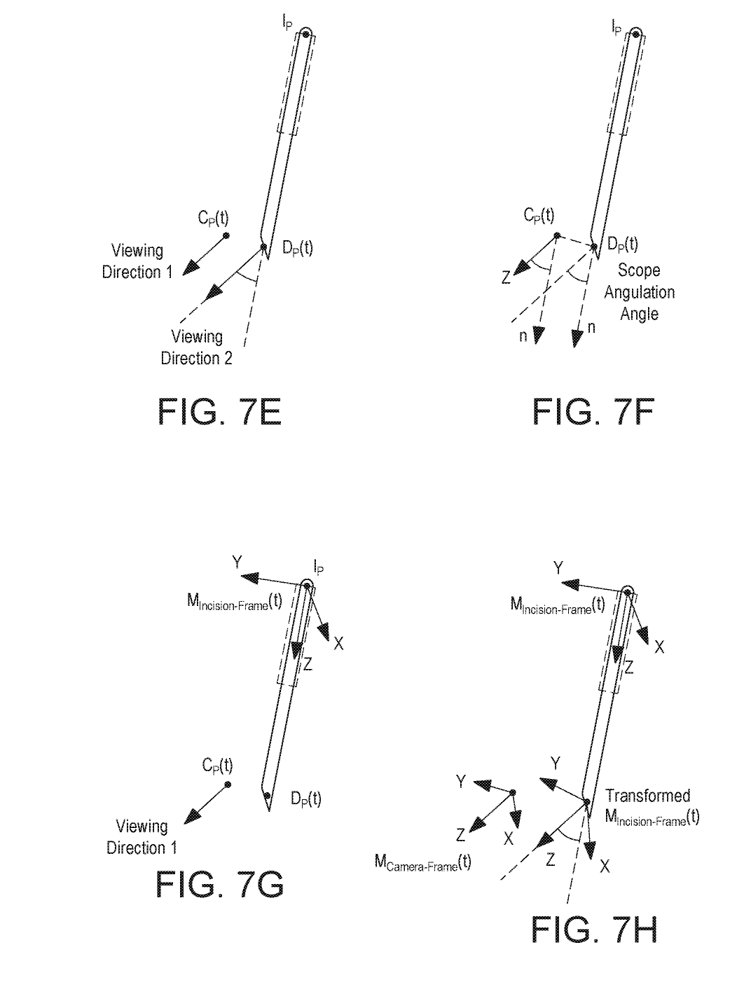

For the articulated laparoscope 200a (FIG. 3A), since the origin of the incision frame M_{Incision-Frame}(t₀) remains constant, the incision frame M_{Incision-Frame}(t) may be represented by point I_{P} and measured with respect to the robot base frame 600. The newly computed camera frame M_{Camera-Frame}(t) is represented by camera point Cₚ(t) along the ‘Z’ axis (FIG. 7A) and is measured with respect to the robot base frame 600, the ‘Z’ axis of the camera frame M_{Camera-Frame}(t) representing the viewing direction. Subsequently, the interface workstation 120 computes the R_{Articulation}(t), the camera frame M_{Camera-Frame}(t) being defined with respect to the robot base frame 600. As illustrated by FIG. 7B, the ‘X’ axis coincides with the camera's viewing direction, the ‘Z’ axis is orthogonal to the ‘X’ axis, and the vector is defined by the end point Iₚ and Cₚ(t). The ‘Y’ axis is computed as a cross product of the ‘Z’ axis and the X′ axis. Further, the points Iₚ and Cₚ(t) are defined with respect to the newly defined camera frame M_{Camera-Frame}(t) such that the points Iₚ and Cₚ(t) become (I_{Px}(t), I_{Py}(t), 0) and (0, 0, 0), respectively (FIG. 7B). As the angle R_{Articulation}(t) remains the same in both the robot base frame 600 and the newly computed camera frame M_{Camera-Frame}(t), the angle R_{Articulation}(t) is computed from the following equation:

I_{Px}(t)sin(R_{Articulation}(t))−I_{Py}(t)cos(R_{Articulation}(t))+(L/R_{Articulation}(t))(cos(R_{Articulation}(t))−1)=0 (3)

wherein ‘L’ denotes the constant Articulated Section Length.

After the R_{Articulation}(t) has been computed, the head tracking system 130 communicates with the interface workstation 120 such that the interface workstation 120 may compute the insertion length L_{Insertion}(t) and the incision frame M_{Incision-Frame}(t) (FIG. 7C). A point A_{P}(t) is computed along the opposite viewing direction and at a distance using the following equation:

(L/R_{Articulation}(t))tan(R_{Articulation}(t)/2) (4)

The insertion length Lᵢₙₛₑᵣₜᵢₒₙ₍ₜ₎ is then computed as the difference between the length of line segment I_{P}A_{P}(t) minus the length of line segment Cₚ(t)A_{P}(t), wherein the incision frame M_{Incision-Frame}(t) is defined by the incision point Iₚ, the ‘Z’ direction is defined by the vector pointing from Iₚ to A_{P}(t), the ‘X’ axis is orthogonal to the plane defined by points Iₚ, A_{P}(t), and Cₚ(t), and the ‘Y’ axis is computed as a cross product of the ‘Z’ and the ‘X’ axis (FIG. 7C). Finally R_{Screen}(t) is computed, wherein R_{Screen}(t) is defined as the angle substituted between the ‘X’ axis of the camera frame M_{Camera-Frame}(t) with ‘X’ axis of transformed incision frame M_{Incision-Frame}(t) (FIG. 7D).

For the angulated laparoscope 200b (FIG. 3B), the origin of the incision frame M_{Incision-Frame}(t₀) remains constant. The incision frame M_{Incision-Frame}(t) may be represented by point I_{P} and measured with respect to the robot base frame 600. The origin of the newly computed camera frame M_{Camera-Frame}(t) is represented by camera point Cₚ(t) (FIG. 7E) and is measured with respect to the robot base frame 600. The ‘Z’ axis of the camera frame M_{Camera-Frame}(t) represents the “Viewing Direction 1” requested by the human operator. The “Viewing Direction 2” represents the feasible viewing direction of the angulated laparoscope with camera 150 positioned at the distal point D_{P}(t). It should be noted that in an ideal scenario D_{P}(t) should coincide with C_{P}(t) and both the viewing directions should be collinear. However, due to the rigid nature and limited possible laparoscope positions and orientations, the points and the directions may not coincide.

Referring to FIG. 7F, subsequently, the interface workstation 120 computes the Lᵢₙₛₑᵣₜᵢₒₙ(t). In order for the vectors representing viewing directions to be collinear and the points D_{P}(t) and C_{P}(t) to have minimum distance, the line segment D_{P}(t)I_{P} should be orthogonal to D_{P}(t)C_{P}(t), as illustrated in FIG. 7F. A unit directional vector ‘n’ is defined by rotating the ‘Z’ axis of M_{Camera-Frame}(t) (which represents the viewing direction 1) by laparoscope angulation angle along the axis orthogonal to C_{P}(t), I_{P}, and ‘Z’ axis. L_{Insertion}(t) is computed by using vector computations as follows:

\vec{Dp(t)}=\vec{Ip}+(\vec{Cp(t)}−\vec{Ip}).n)n (5)

If ∥\vec{Dp(t)}−\vec{Cp(t)}∥>μ (6)

then \vec{Dp(t)}=Cp(t)+μ(Dp(t)−\vec{Cp(t)})/∥\vec{Dp(t)}−\vec{Cp(t)}∥ (7)

L_{Insertion(t)}=∥\vec{Dp(t)}−\vec{Ip}∥ (8)

Where μ is the maximum permissible distance defined by the operator for movement of the laparoscope's distal point D_{P}(t) from the point C_{P}(t) represented by the operator's head-motion M_{Camera-Frame}(t).

After the L_{Insertion}(t) has been computed, the head tracking system 130 communicates with the interface workstation 120 such that the interface workstation 120 may compute the incision frame M_{Incision-Frame}(t) (FIG. 7G), wherein the incision frame M_{Incision-Frame}(t) is defined by the incision point Iₚ, the ‘Z’ direction is defined by the vector pointing from Iₚ to D_{P}(t), the ‘X’ axis is orthogonal to Cₚ(t), IP and Viewing Direction 1, i.e., cross product of vector defined by points Cₚ(t) and Iₚ with Viewing Direction 1. ‘Y’ axis is computed as a cross product of the ‘Z’ and the ‘X’. Finally, R_{Screen}(t) is computed, wherein R_{Screen}(t) is defined as the angle substituted between the ‘X’ axis of the camera frame M_{Camera-Frame}(t) projected on the XY plane of transformed incision frame M_{Incision-Frame}(t) with ‘X’ axis of transformed incision frame M_{Incision-Frame}(t).

Regardless of whether the articulated laparoscope 200a, the angulated laparoscope 200b, or the zero-degree laparoscope 200c is used, the interface workstation 120 then sends the new computed rotational angle R_{Screen}(t) to the video processing system 140 to rotate the video (Step 846). The interface workstation 120 sends the new computed configuration parameters f(t) to the robotic laparoscope holder 110, such as to move the camera 150 to the desired position (Step 848). Once the robotic laparoscope holder 110 has been moved to the new position, the status of the clutch switch 180 is checked and the process continues as described herein until the surgical procedure is complete. If the procedure has not been completed, the laparoscope 200 can be moved away from the cannula 220 (Step 852). The medical professional removes the cannula 220 from the patient's body, the incision is closed (Step 854), and commands are sent to switch off the robotic laparoscope holder 110, the head tracking system 130, and the video processing system 140 (Steps 832-836).

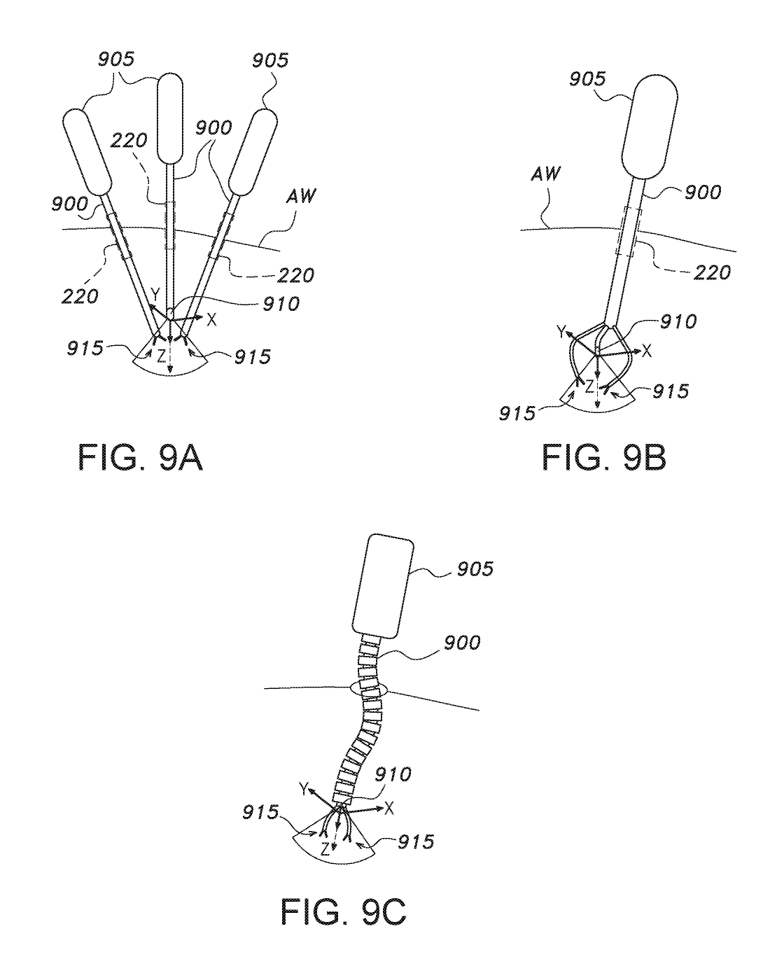

Alternatively, the system for camera control in robotic and laparoscopic surgery 100 may be controlled remotely (e.g. telemanipulated surgical systems as shown in FIGS. 9A through 9C). The system 100 may include at least one robotic arm 900 in communication with a surgical robot (not shown), the at least one robotic arm 900 operatively engaged with a robot base 905. Each robotic arm 900 may include a camera 910 positioned at the end thereof, opposing the robot base 905, and/or a plurality of tooltips 915 (desirably two tooltips 915). The operator may control the position of the camera 910 and, in turn, the camera frame, via his/her head movements, as described herein, leaving the hands free to manipulate the at least one robotic arm 900 and corresponding tooltips 915 via a hand console (not shown).

For example, the operator may operate on tissue using robotic tooltips 915 and, at the same time, view the tool-tissue interaction with the camera 910 affixed to the robotic arm 900. The integration of the control of the tooltips 915 and the camera 910 may allow independent camera control; thereby allowing the hand-held console to be dedicated to the control of the tooltips 915. Such a configuration may allow the simultaneous control of both the camera 910 and tooltips 915 utilized by the operator during the surgical procedure.

The system 100 may, for example, include one robotic arm 900 with the camera 910, and two robotic arms 900 having tooltips 915, such that the surgical procedure requires making three incisions as illustrated in FIG. 9A. The system 100 may include one robotic arm 900 with the camera 910 as well as the tooltips 915, such that the surgical procedure only requires making a single incision as illustrated in FIG. 9B. The system 100 may include one flexible robotic arm 900 with the camera 910 as well as the tooltips 915, such that the surgical procedure only requires making a single incision or using a natural orifice, as illustrated in FIG. 9C.

It is to be understood that the present invention is not limited to the embodiments described above, but encompasses any and all embodiments within the scope of the following claims.

We claim:

1. A system for camera control in robotic and laparoscopic surgery, comprising:
a head tracking system for tracking movements of a head of a human operator, the head tracking system including at least one optical marker for selective attachment to the operator head and an optical tracker configured for detecting movement of the at least one optical marker and transmitting a corresponding sensor signal;
a laparoscope including a shaft having a tip end and a camera disposed at the tip end;
a robotic laparoscope holder selectively connected to the laparoscope, the robotic laparoscope including at least one servomechanism for moving and manipulating the holder and a servo control system;
an interface workstation having:
a processor, and
inputs connecting the sensor signal and the servo control system signals to the processor;
a display;
a video processing system for rendering output from the camera onto the display; and
a clutch switch for activating and deactivating the system for camera control.

2. The system for camera control according to claim 1, wherein the shaft includes an articulating distal portion.

3. The system for camera control according to claim 1, wherein the at least one optical marker is positioned on a band, the band being positioned on the head of the human operator.

4. The system for camera control according to claim 1, wherein the laparoscope is selected from the group consisting of a zero degree laparoscope, an angulated laparoscope, and an articulated laparoscope.

5. The system for camera control according to claim 1, wherein the clutch switch is selected from the group consisting of a foot pedal, a button, a voice activated receiver, and a combination thereof.

6. The system for camera control according to claim 1, wherein the laparoscope includes a proximal portion having a plurality of knobs configured for moving the shaft of the laparoscope.

7. A method for camera control in robotic and laparoscopic surgery, comprising the steps of:
providing a system for camera control in robotic and laparoscopic surgery, the system including
a head tracking system for tracking movements of a head of a human operator, the head tracking system including at least one optical marker for selective attachment to the operator head and an optical tracker configured for detecting movement of the at least one optical marker and transmitting a corresponding sensor signal;
a laparoscope including a shaft having a tip end and a camera disposed at the tip end;
a robotic laparoscope holder selectively connected to the laparoscope, the robotic laparoscope including at least one servomechanism for moving and manipulating the holder and a servo control system;
an interface workstation having:
a processor, and
inputs connecting the sensor signal and the servo control system signals to the processor;
a display;
a video processing system for rendering output from the camera onto the display; and
a clutch switch for activating and deactivating the system for camera control;
inserting the laparoscope into a patients' body;
attaching the at least one optical marker to the head of the human operator;
tracking head movements of the human operator of the system using the head tracking system;
receiving tracking signals from the head tracking system at the interface workstation;
receiving actuating signals from the interface workstation at the robotic laparoscope holder to move the camera in an operating field to correspond with the head movements of the operator;
receiving and processing video from the camera at the video processing system; and
displaying the processed video on the display.

8. The method for camera control according to claim 7, wherein the at least one optical marker is positioned on a band and the band is positioned on the head of the operator.

9. The method for camera control according to claim 7, wherein a servo control system of the robotic laparoscope holder receives the actuating signals.

10. The method for camera control according to claim 7, wherein the laparoscope is selected from the group consisting of a zero degree laparoscope, an angulated laparoscope, and an articulated laparoscope.

11. The method for camera control according to claim 7, further comprising receiving a current rotational angle from the video processing system at the interface workstation.

12. The method for camera control according to claim 7, further comprising receiving current configuration parameters at the interface workstation measured from actuator states of the robotic scope holder.

13. The method for camera control according to claim 7, further comprising receiving new configuration parameters at the robotic scope holder to actuate the robotic scope holder and move the laparoscope.

14. The method for camera control according to claim 7, further comprising receiving a rotated video stream of the operating field at the display from the video processing system.

15. A system for camera control in robotic and laparoscopic surgery, comprising:
a head tracking system for tracking movements of a head of a human operator, the head tracking system including at least one optical marker for selective attachment to the operator head and an optical tracker configured for detecting movement of the at least one optical marker and transmitting a corresponding sensor signal;
a surgical robot including at least one robot base and at least one robotic arm attached to a respective robot base, each robotic arm including a camera and/or one or more tooltips at an end thereof, the robot base being configured for communicating with a hand console for selectively controlling the movement of the at least one robotic arm and each of the tooltips;
an interface workstation having:
a processor, and
inputs connecting the sensor signal and the servo control system signals to the processor;
a display;
a video processing system for rendering output from the camera onto the display; and
a clutch switch for activating and deactivating the system for camera control.

16. The system for camera control according to claim 15, wherein the surgical robot comprises one robot base and one robotic arm, the robotic arm including both a camera and one or more tooltips.

17. The system for camera control according to claim 16, wherein the robotic arm is flexible.

18. The system for camera control according to claim 15, wherein the surgical robot comprises three robot bases and one robotic arm attached to each robot base.

19. The system for camera control according to claim 18, wherein a first one of the robotic arms includes a camera and second and third ones of the robotic arms include one more tool tips.
